(19) United States
(12) Patent Application Publication
(10) Pub. No.: US 2007/0112274 A1
(43) Pub. Date: May 17, 2007

(54) Wireless communication system for pressure monitoring

(75) Inventors: Harold A. Heitzmann, Irvine, CA (US); Charles R. Mooney, Costa Mesa, CA (US); Ann B. Yadlowsky, Irvine, CA (US)

Correspondence Address: INSKEEP INTELLECTUAL PROPERTY GROUP, INC, 2281 W. 190TH STREET, SUITE 200, TORRANCE, CA, 90504, US

(73) Assignee: Edwards Lifesciences Corporation

(21) Appl. No.: 11/292,872

(22) Filed: Dec. 2, 2005

Related U.S. Application Data
(60) Provisional application No. 60/736,428, filed on Nov. 14, 2005.
(60) Provisional application No. 60/736,408, filed on Nov. 14, 2005.

(51) Int. Cl. A61B 5/02

(52) U.S. Cl. 600/485; 128/903

(57) ABSTRACT

In one embodiment, the present invention provides a wireless communication system for use with a blood pressure monitor system. The wireless communication system includes a portable unit that connects to a typical pressure transducer and a monitor interface unit that connects to a typical vital signs monitor. The portable unit obtains a pressure reading from the transducer by providing an excitation voltage to the transducer, then wirelessly transmitting the pressure data to the monitor interface unit. The monitor interface unit measures the excitation voltage supplied by the vital signs monitor to supply the pressure reading in a format recognizable by the vital signs monitor.

### RELATED APPLICATIONS

This application claims priority to U.S. Provisional Application Ser. No. 60/736,428, filed Nov. 14, 2005 entitled Wireless Communication System For Pressure Monitoring; and U.S. Provisional Application Ser. No. 60/736,408, filed Nov. 14, 2005 entitled Wireless Communication Protocol For A Medical Sensor System, and are hereby incorporated by reference.

### BACKGROUND OF THE INVENTION

The measurement of blood pressure is an important technique used by medical personnel for diagnosing and treating a wide range of injuries and conditions. By measuring and especially monitoring a patient's blood pressure, medical personnel can be alerted to problems at an early stage, increasing the likelihood of successful treatment.

While indirect methods of blood pressure monitoring, such as with a pressure cuff and stethoscope, are often desired for quick pressure readings, these methods can be inaccurate by as much as 10 percent, making them undesirable for longer term blood pressure monitoring of more critical patients. Consequently, direct blood pressure monitoring methods are preferred for patients with serious or critical conditions due to their improved accuracy and easier long-term implementation.

The most popular direct blood pressure monitoring method is performed through catheterization, in which a fluid-filled catheter is inserted into a patient at a desired location, such as within a blood vessel. The catheter is filled with a solution, such as saline, and is connected via a tube to a pressure transducer. As the blood pressure within the patient changes, the pressure on the solution within the tube changes proportionally, allowing the connected transducer to accurately measure the pressure within the patient. The pressure transducer is in turn connected to a vital signs monitor which displays the blood pressure readings to the medical personnel. A representative pressure transducer can be seen in U.S. Pat. No. 4,576,181, the contents of which are hereby incorporated by reference.

Typically, transducers have utilized a pressure responsive diaphragm mechanically coupled to piezo-resistive strain gauges arranged in a Wheatstone bridge arrangement. In this respect, the amount of strain placed on the strain gauge can be determined by applying an excitation voltage to the Wheatstone bridge arrangement, then monitoring the output voltage of the bridge. Thus, as the strain varies, the output voltage from the transducer also varies proportionately.

The vital signs monitor connected to the pressure transducer is responsible for providing this excitation voltage to the bridge arrangement and measuring the output voltage to determine the blood pressure within the patient. Currently, most medical device manufacturers recognize a standard in the proportionality of the excitation voltage provided to a transducer and the output voltage in which five microvolts of signal per volt of excitation voltage is equivalent to one millimeter of mercury applied pressure. This standard is also known as standard BP22 “Blood Pressure Transducers” from the Association for the Advancement of Medical Instrumentation (AAMI). The widespread use of this standard allows sensors from many different manufacturers to be interchanged with monitors from other manufactures, enabling the user the flexibility to mix and match components as desired.

One disadvantage to these systems is the cumbersome cable connecting the transducer to the vital signs monitor. These cords can easily tangle, can accidentally pull out from the vital signs monitor, and can be easily confused when multiple pressure monitoring lines are used. Further, the length of these cords limits the distance the patient can move from the vital signs monitor and must be disconnected and secured when a patient is transported within the hospital.

Currently, some wireless transducer products are available, eliminating the use of a cord between the transducer and a visual display. For example, some wireless pressure transducers are available from Memscap, which transmit sensor data to a computer. However, these wireless transducer systems have integrated permanent transducers and wireless functionality to communicate with only a remote personal computer. In this respect, the current wireless transducer systems cannot connect to standard transducers or standard vital signs monitors. Since vital signs monitors are integrated with hospital information systems and represent a significant expense, hospitals are reluctant to switch to these wireless systems which would require the use of only that company's transducer system equipment.

The most common wireless sensor system currently available in some hospitals are wireless ECG transmitters and monitors. ECG telemetry utilize a standard method which transmits. from a portable patient-attached module to a hospital infrastructure, such as a dedicated network of antennas and display monitors. However, unlike invasive blood pressure, ECG does not utilize an artificial transducer or an excitation voltage.

What is needed is a wireless pressure transducer system that can easily connect with the vital signs monitors and ordinary transducers used by many hospitals today.

### OBJECTS AND SUMMARY OF THE INVENTION

It is an object of the present invention to overcome the limitations of the prior art.

It is another object of the present invention to provide a wireless communication system for a pressure transducer system.

It is another object of the present invention to provide a wireless communication system that can function with most pressure transducer systems currently used in hospitals.

It is another object of the present invention to provide a wireless communication system that reduces errors introduced by electrical signal measurement and reproduction.

The present invention attempts to achieve these objects, in one embodiment, by providing a wireless communication system for use with a vital signs monitor system. The wireless communication system includes a portable unit that connects to a typical pressure transducer and a monitor interface unit that connects to a typical vital signs monitor. The portable unit obtains a pressure reading from the transducer by providing an excitation voltage to the transducer, digitizing the output, and then wirelessly transmiting the pressure data to the monitor interface unit. The monitor interface unit receives the digitized voltage supplied by the portable unit and converts the pressure data into a format recognizable by the vital signs monitor.

### BRIEF DESCRIPTION OF THE DRAWINGS

FIG. 1 illustrates a wireless communication system for a vital signs system according to the present invention;

FIG. 2 illustrates a conceptual view of a portable unit according to the present invention;

FIG. 3 illustrates a conceptual view of a monitor interface unit according to the present invention;

FIG. 4 illustrates a conceptual view of a communications system according to the present invention;

FIG. 5 illustrates a conceptual view of a communications system according to the present invention;

FIG. 6 illustrates a conceptual view of a monitor signal conditioning unit according to the present invention;

FIG. 7 illustrates a conceptual view of a multiplying digital to analog converter circuit according to the present invention,

FIG. 8 illustrates a conceptual view of an active bridge drive circuit according to the present invention,

FIG. 9 illustrates a conceptual view of a synthetic bridge circuit according to the present invention,

FIG. 10 illustrates a conceptual view of a load adjustment circuit according to the present invention;

FIG. 11 illustrates a wireless communications system for a vital signs monitor according to the present invention; and

FIG. 12 illustrates a wireless communications system for a vital signs monitor according to the present invention.

### DETAILED DESCRIPTION OF THE INVENTION

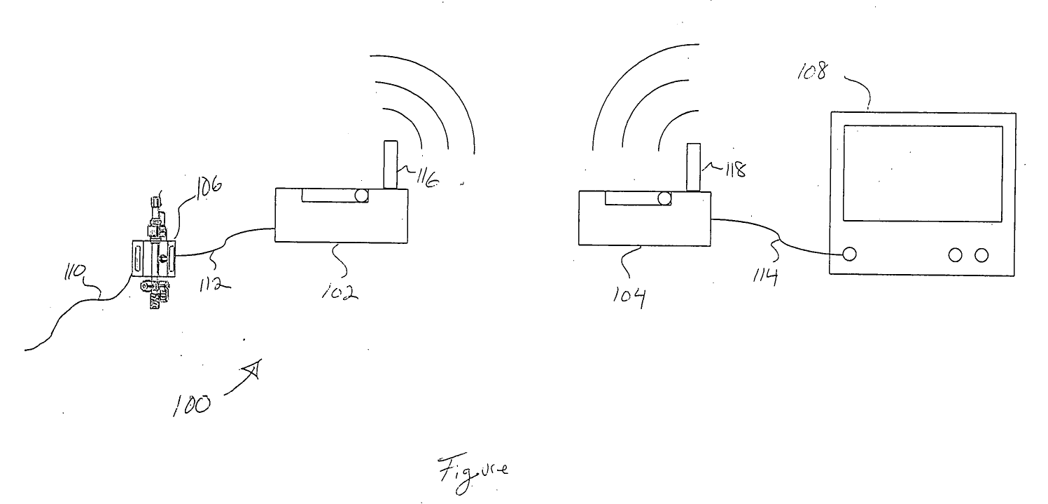

FIG. 1 illustrates a preferred embodiment of a wireless pressure system 100 according to the present invention that can communicate data between a standard pressure transducer 106 (e.g. compliant with the previously described BP22 standard) and a standard vital signs monitor 108 (e.g. compliant with the previously described BP22 standard). More specifically, the wireless pressure system 100 includes a portable unit 102 that provides an excitation voltage to the transducer 106 to receive an output voltage that is proportional to the pressure of a catheter 110. The portable unit 102 digitizes this pressure data, then transmits that data to a monitor interface unit 104 which emulates a corresponding output voltage to the vital signs monitor 108. Consequently, the vital signs monitor 108 receives and displays a signal from the monitor interface unit 104 which corresponds to the actual pressure measured by the portable unit 102, allowing the user to connect and therefore make use of a variety of transducers 106 and vital signs monitors 108 that are compliant with a standard (such as BP22).

As seen in FIG. 1, a standard pressure transducer 106 can be used according to the present invention, preferably supporting the 5 microvolts per volt of excitation voltage standard (5 microvolts/V_{EX}/mmHg). This pressure transducer 106 is connected to a catheter line 110 leading to the interior of a patient, allowing the pressure transducer 106 to be in fluid communication with the cardiovasculature of the patient.

Additionally, a standard vital signs monitor 108 that also supports the 5 microvolts/V_{EX}/mmHg BP22 standard can be used according to the present invention. While the voltage proportion is standardized, the excitation voltage (i.e. the electricity provided for excitation purposes) provided by different manufacturers widely vary in format such as voltage magnitude, timing (e.g. AC or DC), and other characteristics, and therefore the transducers and other equipment that connect to these monitors must be capable of handling the excitation voltage provided. For example, Table 1 illustrates examples of BP22 compliant monitors and some selected characteristics of their excitation voltage.

TABLE 1

| | Excitation | Excitation | Excitation |
|---|---|---|---|
| | Type (AC, | Voltage | Frequency/ |
| Monitor/Module | DC, Pulsed) | (nominal) | Duty |
| Datascope Passport XG | DC | 5.0 VDC | n/a |
| GE/Marquette Solar 8000 monitor & Tram | DC | 5.0 VDC | n/a |
| 450SL module | | | |
| GE Solar 8000 M monitor & Tramrac 4A | DC | 5.0 VDC | n/a |
| chassis & Tram 450SL module | | | |
| GE/Marquette Eagle 3000 | DC | 5.0 VDC | n/a |
| GE Dash 4000 | DC | 5.0 VDC | n/a |
| MDE Escort | DC | 5.0 VDC | n/a |
| MDE Escort II (model 20100) | DC | 5.0 VDC | n/a |
| MDE Escort Prism (model 20403) | DC | 5.0 VDC | n/a |
| Medtronic Lifepak 12 | DC | 4.9 VDC | n/a |
| Philips/HP Merlin & M1006A module | AC | 3.6 Vrms | 2.4 KHz |
| Philips/HP Merlin & M1006B module | DC | 5.0 VDC | n/a |
| Philips/HP Merlin & M1006B module (new style) | DC | 5.0 VDC | n/a |
| Philips M3046A & M3000A module | DC | 5.0 VDC | n/a |
| Philips Omnicare 24 & M1041A chassis & | AC | 3.6 Vrms | 2.4 KHz |
| M1006A module | | | |
| Philips Omnicare 24 & M1041A chassis & | DC | 5.0 VDC | n/a |
| M1006B module | | | |
| Spacelabs Ultraview 1050 | DC | 4.0 VDC | n/a |
| Spacelabs 90308 | DC | 4.1 VDC | n/a |
| Spacelabs 90308 & 90431 chassis/module | DC | 4.4 VDC | n/a |
| Welch Allyn Propak CS (model 244) | Pulsed | 0-5 V | 170 Hz/90% |

The wireless pressure system 100 wirelessly couples the transducer 106 with the vital signs monitor 108 by preferably emulating the 5 microvolts/V_{EX}/mmHg standard. More precisely, this emulation includes two discrete actions: emulation of the excitation voltage of the vital signs monitor 108 to the transducer 106 and emulation of the voltage output of the transducer 106 to the vital signs monitor 108.

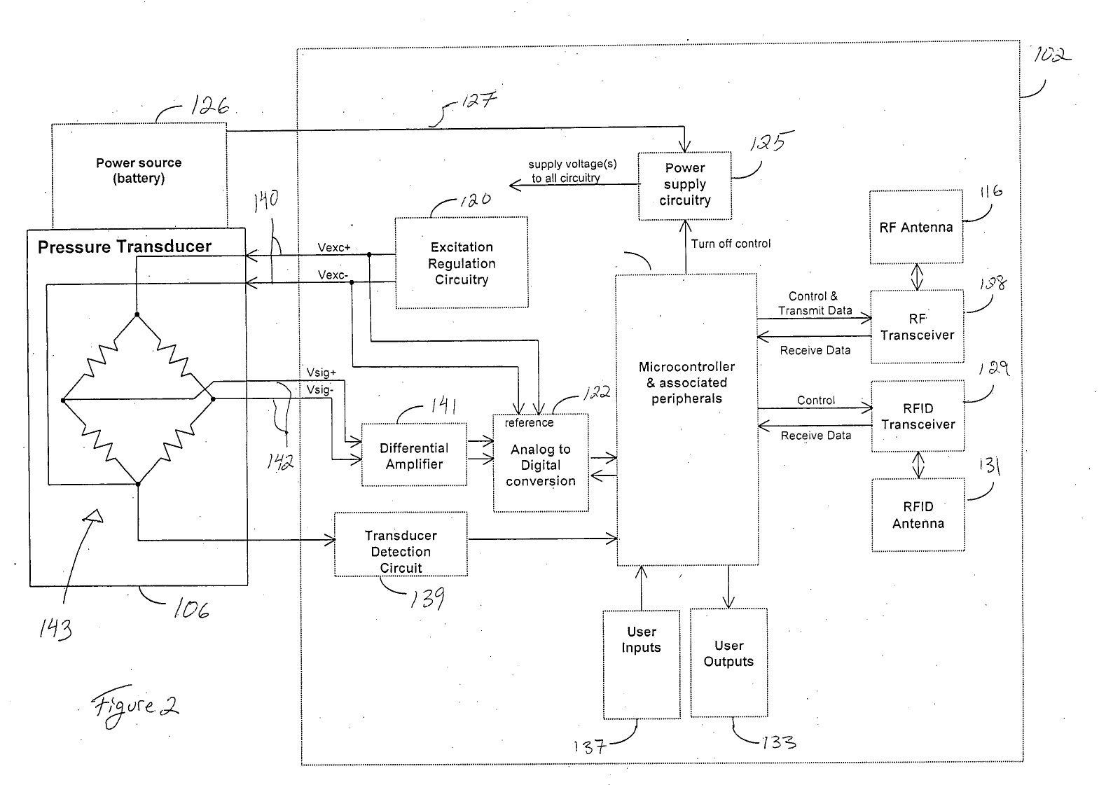

The excitation voltage emulation is performed by the portable unit 102, which is connected to the transducer 106 by power cable 112 (seen in FIG. 1 only). As seen in the schematic drawing of FIG. 2, the portable unit 102 includes a voltage excitation circuit 120 which supplies and regulates an excitation voltage 140 through wires within the power cable 112 to the transducer 106. This voltage excitation circuit 120, along with the other circuits of the portable unit 102, is powered by a power supply circuit 125 which draws its power from an external battery 126, in this case removably secured to the transducer 106.

Since the portable unit 102 ultimately generates a pressure reading in a digital form, the excitation voltage 140 can be in a variety of different formats or voltages. Preferably, excitation voltages 140 with minimal power requirements are preferred to maximize the life of the battery 126. In a preferred embodiment, the excitation voltage 140 is equal to about 1.225 Volts.

As previously described, the excitation voltage 140 travels through a resistive bridge 143 (such as a Wheatstone bridge) within the transducer 106 and provides an output voltage 142 according to the 5 microvolts/V_{EX}/mmHg standard through additional wires in cable 112 to the portable unit 102.

Once in the portable unit 102, the output voltage 142 initially passes through a differential amplifier 141 which “cleans up” the voltage signal by applying amplification and filtering. Next, an analog to digital converter 122 (AD converter) produces a digital value based on both the amplified and filtered output voltage 142 and the original excitation voltage 140 (i.e. a reference voltage), transmitting these digital measurements on to a microcontroller 132. The microcontroller 132 converts these digital voltage values into a pressure reading according to the BP22 standard (5 microvolts/V_{EX}/mmHg standard), for example by using the formula Pressure (mm Hg)=(V_{T}/(Vₑₓ×5 μV))×(mm Hg×V), where Vₑₓ is equal to the excitation voltage 140 and VT is equal to the output voltage 142. In a preferred embodiment the digital value attributed to the pressure reading will be between 0 and 4095. Alternately, the digital value can simply be maintained without conversion, allowing the monitor interface unit 104 to manipulate the digital value appropriately. It should also be understood that a variety of software techniques can be used in this regard so that the monitor interface unit 104 can interpret this digital data and produce an emulated analog transducer signal.

Next, the microcontroller 132 readies this pressure data to be sent to the monitor interface unit 104 by creating a data packet appropriate for wireless transmission. For example, this may include adding time stamp information for the pressure data, CRC error detection data, unit identification data, and other relevant information.

After its assembly, the data packet is communicated to the RF transceiver 128, which transmits the data packet via antenna 116. This wireless RF transceiver 128 may transmit and receive with a variety of different frequencies and protocols, such as radio frequencies, infrared frequencies, Bluetooth protocols, and TDMA protocols.

Thus, by supplying excitation voltage 140 to the transducer 106 and comparing the output voltage 142 to the original excitation voltage 140, the portable unit 102 interacts with the pressure transducer 106 in a similar manner as a vital signs monitor 108, but instead obtains digital pressure data that can be transmitted wirelessly.

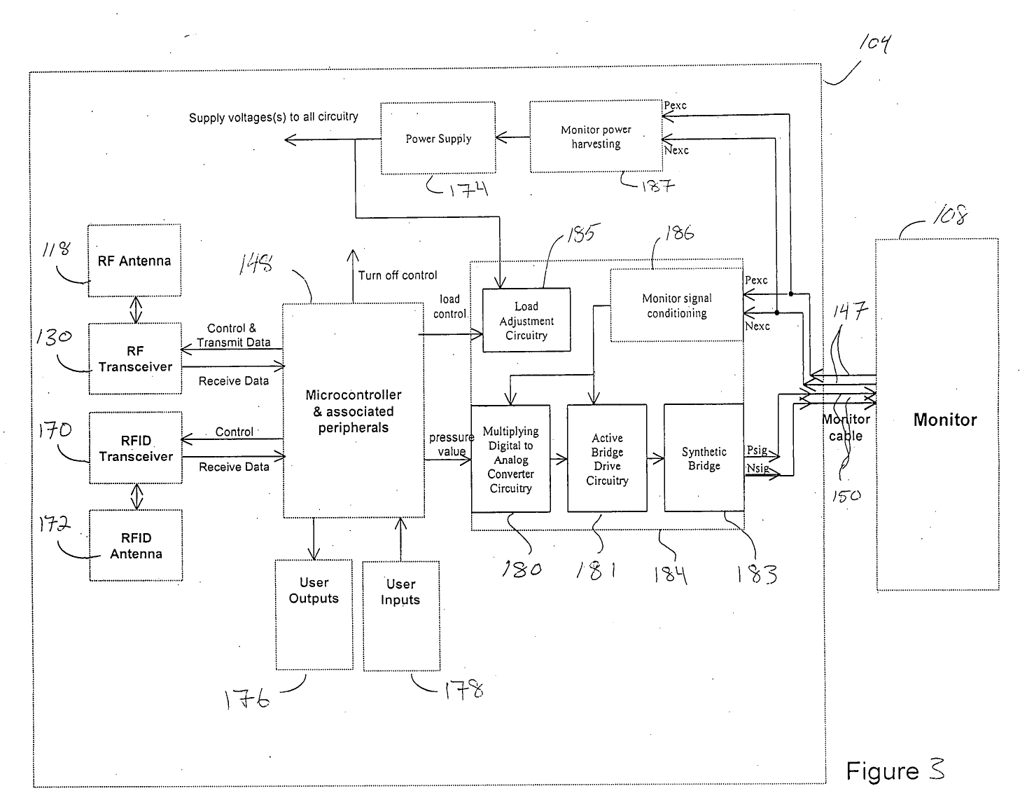

The emulation of the voltage output from the pressure transducer 106 is performed by the monitor interface unit 104, which is connected to the vital signs monitor 108 by cable 114. As seen in the schematic drawing of FIG. 3, the monitor interface unit 104 includes a RF antenna 118 connected to a RF transceiver 130 configured to receive the data packet transmitted by the portable unit 102. Once received, the transceiver 130 sends the received data packet to the microcontroller 148 to extract and process the relevant information, including the pressure data.

To determine the voltage value which is appropriate to communicate the pressure reading to the vital signs monitor 108, the monitor interface unit 104 must also be “aware” of the format (e.g. voltage magnitude, A.C. or D.C., etc.) of the monitor excitation signal 147 that is produced by the vital signs monitor 108, or at least couple to and manipulate this signal 147. As previously discussed, most vital signs monitors supply their own transducer excitation signal 147 and expect a BP22 standardized transducer signal based on the monitor excitation signal 147. Additionally, the monitor excitation signal 147 can also server as a source of power for the monitor interface unit 104, as described in more detail below. In other words, the monitor excitation signal 147 can be used both as a source of power and also as a reference for the transducer emulation circuit.

In the present embodiment, this monitor excitation signal 147 is supplied through isolated wires within cable 114 to a pressure transducer emulation circuit 184 and a monitor power harvesting circuit 187. The monitor power harvesting circuit 187 converts the monitor excitation signal 147 into a format appropriate for use by the circuits of the monitor interface unit 104. Preferably, the monitor power harvesting circuit 187 is responsible for converting AC power, if present, into DC power since DC power is typically used by the circuits and chips of electronic devices. This AC to DC conversion can be achieved, for example, by rectifying the AC power with a diode bridge. Thus, the monitor power harvesting circuit 187 supplies DC power, despite an input power of either AC or DC power from the monitor excitation signal 147. The monitor power harvesting circuit 187 supplies this DC power to a power supply 174 which reduces the voltage to a level appropriate for use by the chips and circuits of the portable unit 102, such as 3.5 volts, then distributes this power to the circuits of the monitor interface unit 104. In this manner, the monitor interface unit 104 can power itself, and therefore all of its circuits exclusively by the excitation signal 147 produced by the vital signs monitor 108. In an alternative preferred embodiment, the power supply circuit 174 can draw power from an A.C. adapter or a battery.

Returning again to FIG. 3, the pressure transducer emulation circuitry 184 includes a multiplying digital to analog converter circuit 180, an active bridge drive circuit 181, a synthetic bridge circuit 183, a load adjustment circuit 185, and a monitor signal conditioning circuit 185, all of which are responsible for converting or “translating” the digital pressure value received from the portable unit 102 into an analog form that the vital signs monitor 108 can read. Preferably, this emulation can be achieved by modifying the monitor excitation signal 147 according the digital pressure data obtained by the portable unit 102, as will be explained in greater detail below. An example of another pressure transducer emulation circuit can be seen in U.S. Pat. No. 5,325,865, the contents of which are hereby incorporated by reference.

The analog emulation process (i.e. producing a pressure signal recognizable to the vital signs monitor 108) allows the excitation signal 147 (represented as “Pexc” and “Nexc” in FIG. 3) to enter the monitor signal conditioning circuit 186 of the emulation circuitry 184. The conditioning circuit 186 accepts the excitation signal 147 and “conditions” this power signal appropriately to be used by the multiplying DA converter circuit 180 and the active bridge drive circuit 181, then supplies these circuits 180 and 181 with the conditioned power signal. More specifically, the conditioning circuit 186 converts the differential voltage signal from the monitor excitation signal 147 (i.e. Pexc and Nexc) into a reference voltage signal, or a voltage signal with a difference relative to the ground of the monitor interface unit 104.

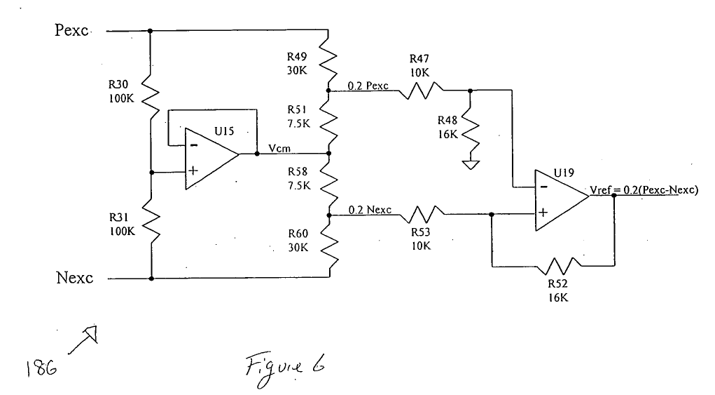

FIG. 6 illustrates a more specific schematic example of a conditioning circuit 186. In this example, resistors R30 and R31 create a reference potential, or virtual ground, halfway between Pexc and Nexc labeled Vcm (for common mode voltage) and driven by amplifier U15. Resistors R49/R51 and R60/R58 form resistor dividers which create the signals 0.2 Pexc and 0.2 Nexc respectively reference to Vcm. These voltages are fed into the amplifier formed by resistors R47, R48, R53, R52, and amplifier U19 (e.g. part number LMC7111 from National Semiconductor Corportation) which is configured to provide unity gain and differential to single ended conversion between the balanced excitation potentials 0.2 Pexc and 0.2 Nexc. The output of this amplifier is the reference input (Vref), which is supplied to the multiplying DA converter circuit 180 and the active bridge drive circuit 181.

The multiplying digital to analog converter circuit 180 accepts and modifies this referenced power signal (Vref in the specific example) according to the digital pressure value obtained from the microcontroller 148. More specifically, the DA converter circuit 180 outputs a differential current that is proportionally lower than the referenced power voltage value, based on the value of the digital pressure value. For example, this conversion can be achieved by first determining the ratio by which the conditioned analog signal should be reduced (e.g. dividing the current digital pressure value by the maximum digital pressure value of the AD converter circuit 122 in the portable unit 102), then reducing the voltage of the referenced analog signal by that ratio (e.g. multiplying the ratio by the value of the referenced analog signal). Generally speaking, this ratio acts as a “conversion factor” for the digital pressure value since the digital value alone is not absolute, but rather an arbitrary digital number that differs with different types of analog to digital circuits. Thus, the DA converter circuit 180 modifies the value of the voltage output proportionally based on this ratio. It should be noted that such conversion factors may differ, depending on how the digital pressure data is provided to the monitor interface unit 104. However, no matter what the format of the digital pressure data is, it can be converted into a form usable by the digital to analog converter circuit 180.

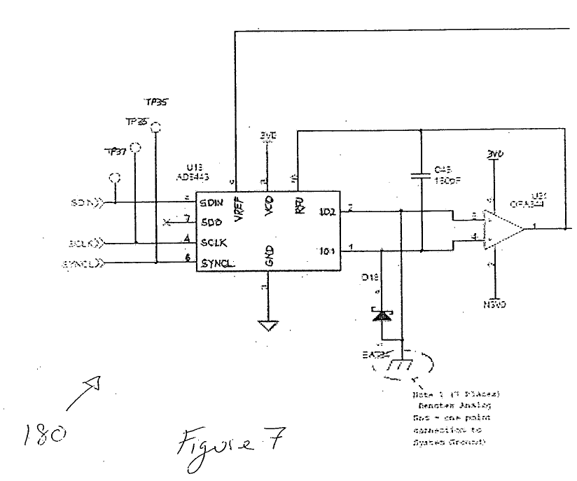

FIG. 7 illustrates a more specific schematic example of an AD5443 digital to analog converter circuit, as produced by Analog devices, Inc. U13 of this specific example operates on a 3V single ended regulated power supply, while U21 operates from a split regulated supply providing +3V and −3V. The reference voltage or Vref is supplied from U19 (LMC7111) as previously discussed in regards to the conditioning circuit 186. Preferably, this circuit is used in a bipolar, four quadrant multiplying design as described in the accompanying data sheet of the AD5443 and as is known in the art.

The “proportioned” reference analog signal is next supplied to the active bridge drive circuit 181 which creates a voltage bridge that “drives” the synthetic bridge 183 to produce the final simulated transducer signal 150. Specifically, the active bridge drive circuit 181 modifies the analog reference signal, for example by summing multiple voltage signals such as the reference signal, the proportioned reference signal and the common mode signal, to achieve an appropriate value for the synthetic bridge 183. The synthetic bridge 183, in turn, attenuates the output voltage of the bridge drive circuit 181, then converts this referenced analog signal back into a differential signal, creating the simulated transducer signal 150.

FIG. 8 illustrates a specific example of an active bridge drive circuit 181 in which an inverting amplifier is formed by resistors R63, R64, and amplifier U20 to invert and attenuate the signal Vcm from the monitor signal conditioning circuit 186 by a factor of 2. Additional inverting is achieved with a summing amplifier formed from resistors R50, R56, R62, R59, and amplifier U20. The output of this circuit can be described by the following formulas: Vbridge=−(0.5Vref+(−Vref*DAC)+2(−0.5Vcm))=−0.5Vref+Vref*DAC+Vcm; OR Vbridge=Vref (DAC−0.5)+Vcm.

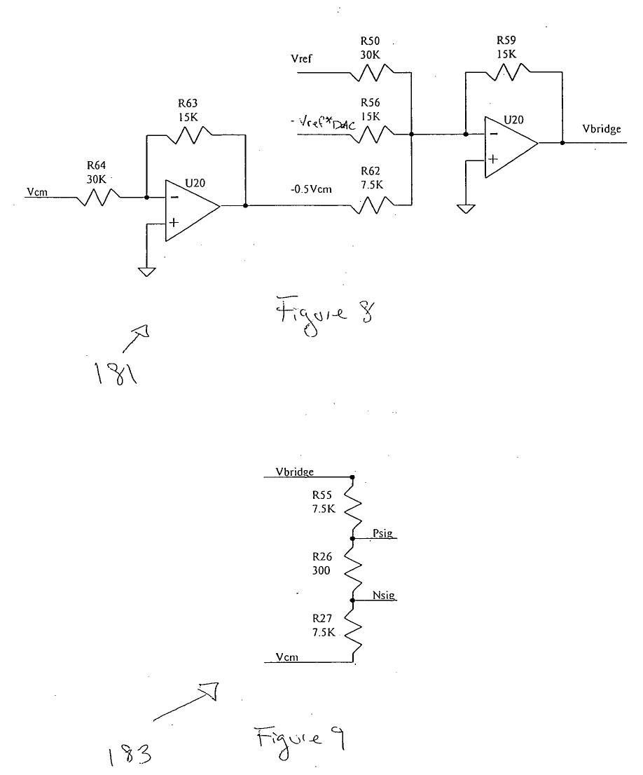

FIG. 9 illustrates a specific example of a synthetic bridge 183 in which the signal from the active bridge circuitry of FIG. 8, Vbridge, is applied to resistor R55, while the common mode voltage, Vcm, is applied to the other side of the bridge at resistor R27, meeting in the middle with resistor R26. The final output, which has been attenuated and differentiated, can be seen as Psig and Nsig, which represents the final simulated transducer signal 150. This final simulated transducer signal 150 can be described in this specific example by the following equations:
Psig−Nsig=(R26/R55+R26+R27))(Vbridge−Vcm)=1/51(Vref(DAC−0.5))
Since Vref=0.2(Pexc−Nexc)
Psig−Nsig=1/255(Pexc−Nexc)(DAC−0.5)

The digital value written to “DAC” in the previous equation can range from 0 to 4095 in the previous specific example. As previously discussed, the differential signal output expected by the vital signs monitor 108 is scaled to 5 μV per volt of excitation per mmHg. Thus, with 1 volt of excitation (Pexc−Nexc=1), a digital value of 4095 (full scale) would correspond to a differential signal (Psig−Nsig) of 0.5/255=0.00196 which is equivalent to 392 mmHg. A digital value of 0 would be equivalent to −392 mmHg. A digital value of 2048 would be equivalent to 0 mmHg.

This simulated transducer signal 150 is supplied by the synthetic bridge 183 through wires within cable 114 (seen only in FIG. 1) to a signal input port on the vital signs monitor 108, allowing the vital signs monitor 108 to process and display the pressure reading from the simulated transducer signal 150. Thus, the vital signs monitor 108 functions as if it was directly connected to and interacting with the transducer 102, when it is actually interacting with the monitor interface unit 104.

The transducer emulation circuit 184 also includes the load adjustment circuitry 185 which allows the microcontroller to emulate a “load” or resistance amount on the monitor excitation signal 147. Specifically, the load adjustment circuitry 185 monitors the load being drawn from the excitation signal 147, and when this load deviates from an amount that would normally be drawn by a typical pressure transducer, the microcontroller 148 causes the circuit 185 to increase or decrease resistance. In this respect, the monitor interface unit 104 draws a similar amount of power in a similar way to a standard pressure transducer. Since some monitors 108 have alarms that may be triggered if the load is outside of a specified range, the load adjustment circuitry 185 can maintain the load at a normal level, preventing false alarms.

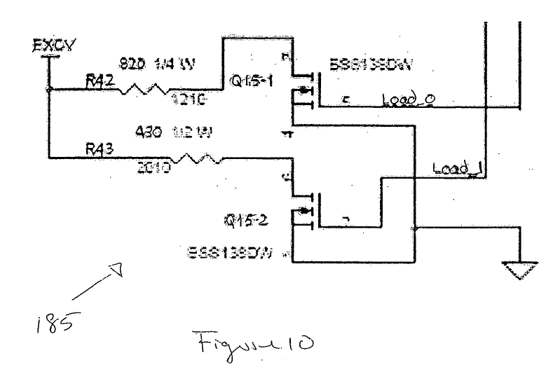

A more specific example of such an emulation circuit can be seen in the schematic illustration in FIG. 10. In this example, the load adjustment circuitry 185 includes switched resistances R42 (820Ω) in series with Q15-1 and R43 (430Ω) in series with Q15-2. The microcontroller 148 signal controlling Q15-1 is “Load 0” while the signal controlling Q15-2 is “Load 1”. If the other loads of the monitor interface unit 104 become too low, the microcontroller 148 can send a signal to turn on either Load 0 or Load 1. Conversely, if the other loads of the monitor interface unit 104 become too high, the microcontroller 148 can send a signal to turn off either load, thus regulating the amount of current drawn by the monitor interface unit 104.

In operation, the user first connects the portable unit 102 to the pressure transducer 106, then connects the monitor interface unit 104 to the vital signs monitor 108. Next, the user activates the portable unit 102 and monitor interface unit 104 and “links” these units 102 and 104 together so that they recognize the RF signals transmitted by each other. In one preferred embodiment, the user can enter a “linking code” into each unit 102 and 104 by way of the user inputs 133 and 178 and the user outputs 137 and 176, as seen in FIGS. 2 and 3 respectively. In an alternative preferred embodiment, an RFID token can be used to transmit a “linking code” to each unit 102 and 104 through the RFID transceivers 129 and 170, and RFID antennas 131 and 172, as seen in FIGS. 2 and 3 respectively. In this respect, the portable unit 102 and the monitor interface unit 104 can use the linking code to identify wireless transmission from each, while ignoring transmissions from nearby, non-linked units. A more detailed discussion of this linking or pairing process can be seen in the concurrently filed and commonly assigned U.S. Provisional Application No. 60/736,408 entitled Wireless Communication Protocol For A Medical Sensor System, filed on Nov. 14, 2005, the contents of which are hereby incorporated by reference.

After recognizing each other, the portable unit 102 begins sending an excitation signal 140 to the pressure transducer 106, measuring and converting the output signal 142 into a digital pressure reading. The microcontroller 132 encodes this pressure data into a data packet suitable for wireless transmission and ultimately transmits this data packet with RF transceiver 128. This process is continually repeated, creating a stream of data packets that are wirelessly transmitted.

The monitor interface unit 104 receives the data packets with transceiver 130 and the microcontroller 148 parses out the relevant data, including the digital pressure values. Each digital pressure value is sent to the pressure transducer emulation circuit 184 which produces an analog signal based on the BP22 standard and communicates this simulated transducer signal 150 back to the vital signs monitor 108. The vital signs monitor 108 interprets the simulated transducer signal 150 as a pressure reading and displays the value according to its functionality.

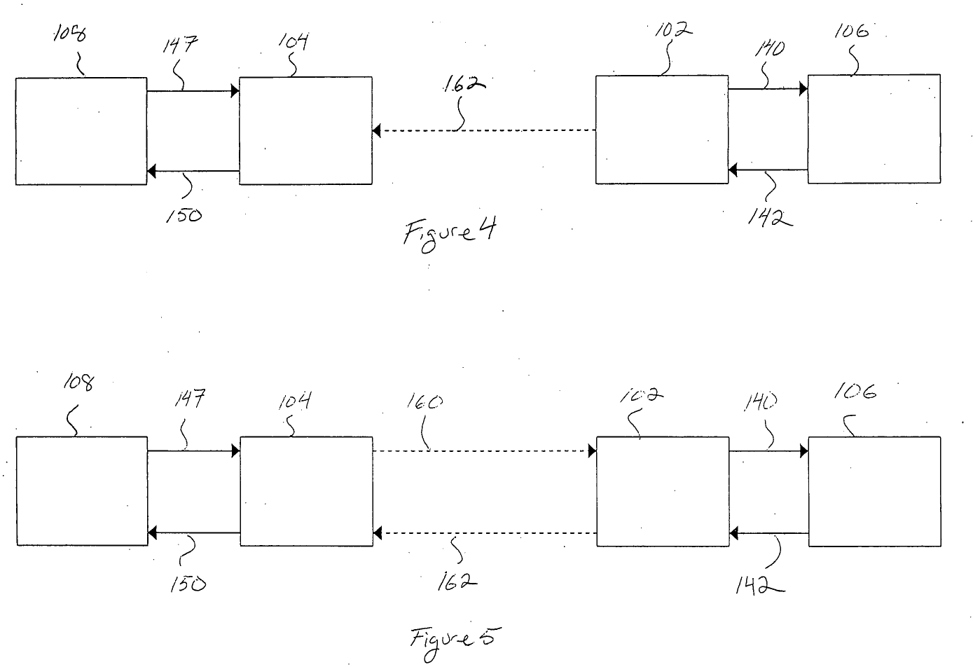

It should be noted that the overall architecture of this preferred embodiment of the wireless pressure system 100 acts to minimize errors that may distort the pressure reading displayed at the vital signs monitor 108 when compared with alternative embodiments. The reasons for this error minimization can be more clearly appreciated by comparing the present embodiment as seen in FIG. 4 with an alternate embodiment as seen in FIG. 5.

In the alternate embodiment of FIG. 5, the monitor excitation signal 147, i.e. the electrical signal delivered by the vital signs monitor 108 to excite the transducer of a typical wired system, is continuously measured and recorded into a data signal which is transmitted over wireless signal 160 to the portable unit 102. Such a continuous measurement may be desired with monitors that, for example, provide pulsing monitor excitation signals 147 and therefore expect a pulsing return signal. The portable unit 102 reads the data within the wireless signal 160 and creates transducer excitation voltage 140 accordingly. The transducer output voltage 142 from the transducer 106 passes back to the portable unit 102 where it's transmitted via a wireless signal 162 to the monitor interface unit 104. Finally, the monitor interface unit 104 generates a simulated transducer signal 150 based on the data sent in the wireless signal 162.

Thus, this alternate embodiment includes multiple measurements and voltage emulations in series, creating a more complex series of steps. Further, the transducer excitation voltage 140 is directly derived from the measurement of the monitor excitation voltage 147. Since almost all electrical measurements and voltage reproductions introduce at least some error or inaccuracy, multiple measurements and reproductions in series may increase these errors, possibly combining and magnifying them. Additionally, emulating the exact monitor excitation signal 140 at the portable unit requires many different circuitries to achieve such a wide range of voltage. Further, a larger battery will typically be necessary since most monitors 108 provide a relatively high excitation voltage 147. In other words, the increased complexity of this embodiment can more easily lead to errors and additional demands on the components of each unit 102 and 104.

In contrast, the preferred embodiment of FIG. 4 functions as previously described in this specification. Namely, the portable unit 102 provides a predetermined transducer excitation voltage 140 to the transducer 106 which is returned to the portable unit 102 by transducer output voltage 142 and ultimately transmitted to the monitor interface unit 104 via wireless signal 162. Thus, the alternate preferred embodiment of FIG. 5 is much more complex when compared with the preferred embodiment of FIG. 4. Specifically, an additional emulation step occurs with the measurement of the monitor excitation signal and the reproduction or emulation of that measurement with the transducer excitation voltage 140. In other words, the transducer excitation voltage 140 in FIG. 4 is not derived from measurements in the monitor interface unit 104 which can allow for more accurate measurement. Therefore, the preferred embodiment of FIG. 4 is much less likely to produce errors or increase pre-existing errors.

In some patient monitoring systems, multiple sensors can be connected to a single vital signs monitor. Instead of including many different types of sensor ports on a single vital signs monitor (i.e. one blood pressure sensor port, one EKG sensor port, etc.), some monitors provide a plurality of generic ports into which different vital signs sensors can be connected. Since each sensor may have a different physical connection port, different power requirement, and a different data transmission scheme, these vital signs monitors rely on interface modules to “interface” between the generic ports of the monitor and one particular sensor type (e.g. a blood pressure transducer).

Thus, the interface module accommodates the specific connection, power, and data requirements of the sensor, then transmits the sensor data on to vital signs monitor. In this respect, interface modules can greatly simplify the amount of equipment used in a typical hospital room by allowing many different types of patient sensors to connect and therefore display on a single vital signs monitor.

For example, an interface module for a wired pressure transducer may provide an excitation signal to the pressure transducer while measuring the output voltage of the transducer. The interface module may then convert this pressure reading into a proprietary format understood by the vital signs monitor and further communicate this data via one of the generic ports on the vital signs monitor. The interface module may also provide additional information to the vital signs monitor to facilitate proper display of the data, such as the measurement units, how to graph the data, or critical sensor levels that signal an alarm (e.g. very low blood pressure may automatically cause an alarm to sound on the vital signs monitor).

In order to accommodate different sensor types, each interface module must be customized to work with a specific type (and sometimes brand) of sensor. Thus, different sensor types require a different interface module. In one respect, such customizations are directed to including a port on the interface module that will connect to a port or connector on the sensor. In other words, the sensor must be able to physically plug into the interface module.

In another respect, such customizations are directed to drivers or software specific to each sensor type. These drivers allow the interface module to interpret the raw data received from each sensor and convert it (e.g. with a conversion routine) into a format that can be displayed on a monitor. Additionally, these drivers also provide the communication format of the monitor which allows the interface module to communicate this sensor data in a form understandable to the vital signs monitor. While most processing of the sensor data occurs in the interface module, additional conversions and calculations of the data can also occur in the vital signs monitor.

One example of such an interface module is the Philips M1032A Vuelink Module which can connect to the generic sensor ports of a Philips IntelliVue line of vital sign monitors. More information regarding the Philips Vuelink Module can be found the Vuelink brochure entitled “Vuelink Device Interfacing Module” document number 452298291381 printed in the Netherlands in August of 2003, the contents of which are hereby incorporated by reference. Additional examples and interface module discussion can be seen in U.S. Pat. Nos. 6,477,424 and 5,666,958; and U.S. Pub. No. 2003-0028226, the contents of which are hereby incorporated by reference.

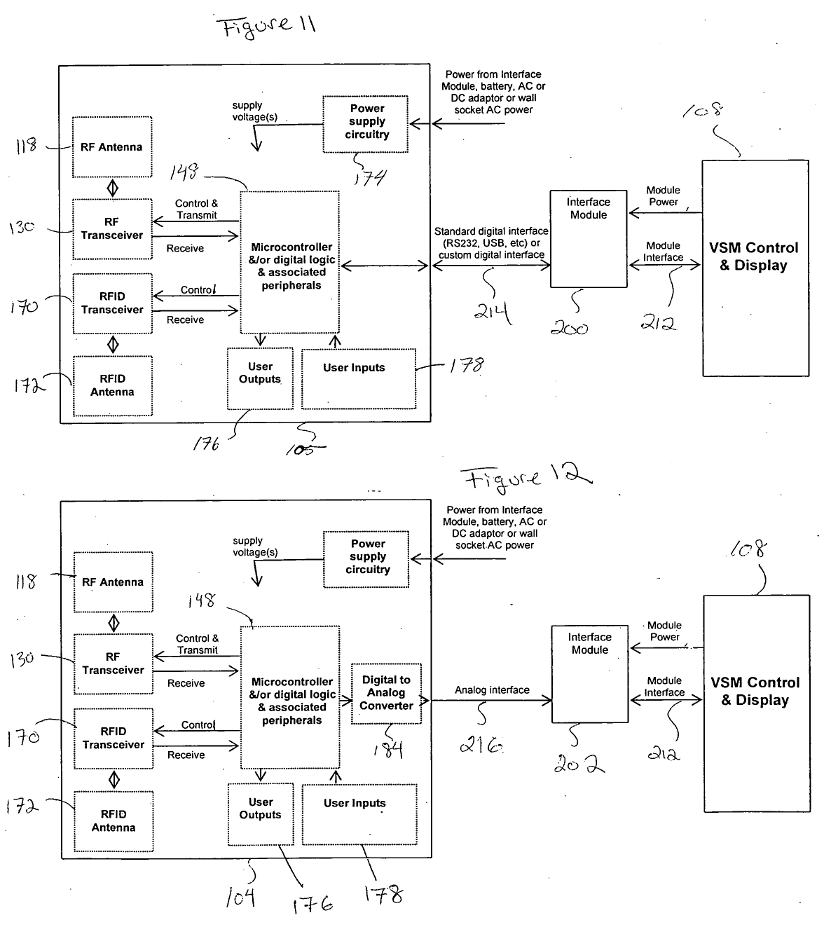

The wireless pressure system 100 described in this specification can be adapted to connect with such an interface module. For example, FIG. 11 illustrates one preferred embodiment that connects to and communicates with such an interface module 200. Specifically, a digital monitor interface unit 105 is provided which is generally similar to the previously described monitor interface unit 104, except this unit 105 outputs a digital pressure signal instead of an emulated analog signal and does this over a digital interface 214 (e.g. a cable between the digital monitor interface unit 105 and the interface module 200). In other words, the monitor interface unit 105 does not convert the digital pressure value back to an analog value, as is the case in the previously described preferred embodiments. Instead, this digital value is transmitted, either in a raw form or a standardized form (i.e. a form understandable to the interface module 200) over the digital interface 214.

The interface module 200 is connected to the digital interface 214 and includes software that can interpret or convert the digital data as a standard pressure value (e.g. mmHg). Since the monitor interface unit 105 can supply digital pressure data in a many different digitals forms, the interface module 200 must understand how this digital data relates to the actual pressure data measured on the patient. In other words, the interface module 200 must know how to convert this digital data into a meaningful form. Preferably, this conversion is fully performed in the interface module 200, however part or all of these interpretations or conversion routines can be performed in the monitor interface unit 105 or in a connected adapter unit.

Since these conversions may vary depending on the make and type of the sensor, the conversion algorithms or routines can be automatically selected based on detection of specific sensors (e.g. plug and play devices) or can be manually selected by way of inputs (buttons, switches, touch screens, etc.). Thus, the conversion routine or algorithm needed for the interface module 200 to “understand” the digital data can be selected for different sensors.

The interface module 200 then sends the appropriate data over monitor interface 212 (e.g. a direct connection, cable, etc.) to cause the digital pressure value to be displayed on the vital signs monitor 108. In this respect, the pressure value remains a digital value after being communicated to the interface module 200, allowing the interface module 200 to appropriately communicate the patient pressure data to the vital signs monitor 108 for display.

In addition to including the ability to display on a specific type of vital signs monitor (e.g. with a specific proprietary monitor driver), the interface module 200 also preferably includes the ability to display sensor data on many different types of vital signs monitors 108 (e.g. by including many different vital signs monitor drivers). In such situations, different monitors may be automatically detected, or a user may select an appropriate monitor from an input (e.g. buttons or switches) on the interface module 200. Additionally, the interface module 200 preferably includes the hardware and software components necessary to connect to standard vital signs monitors, either through a typical display port or through a sensor input port using an emulated sensor signal, as described previously in this specification.

FIG. 12 illustrates another preferred embodiment generally similar to the previously described preferred embodiment, except a monitor interface unit 104 interfaces with an interface module 202 that produces an analog signal. Preferably, the interface module 202 is configured to interact with a pressure transducer by the BP22 standard; however other analog data transmission methods are also acceptable.

The monitor interface unit 104 is connected via analog interface 216 (e.g. a cable) to the interface module 202, allowing the monitor interface unit 104 to provide analog pressure data in a standard format (e.g. BP22) or any proprietary format. The interface module 202 converts this simulated analog pressure signal into an appropriate data format (either analog or digital), then communicates the pressure data over monitor interface 212 to the vital signs monitor 108 (as described in the previous example). In other words, the preferred embodiment of the wireless pressure system 100 initially described in this specification is essentially connected to an interface device and displayed on a vital signs monitor.

While the term interface module has been described in this specification, it should be understood that this term can be more broadly understood to mean any device that can connect between a patient sensor and a vital signs monitor. It should also be understood that there may be a variety of different arrangements that can facilitate connection of the wireless pressure system 100 to a vital signs monitor 108 (i.e. a wireless connection that ultimately results in a pressure display on a vital signs monitor 108). While a few of these arrangements have been described (e.g. a direct connection to the vital signs monitor 108, an interface module, etc.), other arrangements are contemplated as falling within the scope of this invention.

For example, the interface modules 200 and 202 may directly connect to a port on the vital signs monitor 108.

In another example, the interface modules 200 and 202 can be incorporated into monitor interface units 104 and 105 respectively. In this respect, each monitor interface unit 104 or 105 includes a generic connector (to connect to the vital signs monitor) and an interface circuit that is configured or customized to communicate with a specific vital signs monitor 108 or series of monitors from a particular manufacture (i.e. a proprietary communications protocol is used). Thus, the monitor interface units 104 or 105 include the software, drivers, protocols, connection ports, and similar elements that allow these monitor interface units 104 or 105 to directly connect to and interact with the communications bus of the vital sign monitor 108, a vital signs monitor chassis, or a vital signs monitor rack.

Further, such direct connection allows the vital signs monitor 108 to more easily communicate with and control the interface modules 104 and 105. For example, a user can actuate an input device (e.g. buttons) on the vital signs monitor 108 to shut down the monitor interface unit 104 or 105.

This direct connection between the vital signs monitor 108 may also facilitate the communication of other data types to be displayed on the vital signs monitor 108. For example, the monitor interface unit 104 or 105 can transmit wireless signal strength data and a battery level of the portable unit 102, in addition to the pressure data. This additional data can be displayed on the vital signs monitor 108 or used as the basis for alarms (e.g. an audible noise when the battery level of the portable unit 102 is critical).

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A system for measuring blood pressure in a patient comprising:
a pressure transducer;
a first unit connected to said pressure transducer, said first unit generating and wirelessly transmitting a digitized signal representative of said blood pressure;
a second unit in electrical communication to any one of a plurality of different vital signs monitors, said second unit wirelessly receiving said digitized signal and processing said digitized signal so as to generate an analog signal suitable for use with any one of said plurality of different vital signs monitors.

2. A system according to claim 1, wherein said first unit includes an excitation voltage circuit independent of any excitation voltage signal generated by said vital signs monitor.

3. A system according to claim 1, wherein said analog signal suitable for use with any one of a plurality of different vital signs monitors compliant with the Association for the Advancement of Medical Instrumentation Standard BP22 “Blood Pressure Transducers”.

4. A system according to claim 1, wherein said second unit includes a pressure transducer emulation circuitry for receiving and processing said digitized signal from said first unit.

5. A system according to claim 4, wherein said pressure transducer emulation circuitry comprises:
a monitor signal conditioning circuit for generating a reference voltage based on an excitation voltage of said monitor;
a multiplying digital to analog converter circuit for generating an analog signal based on said reference voltage and said digitized signal from said first unit;
an active bridge circuit for generating an active bridge signal based on said analog signal;
a synthetic bridge circuit for generating a differential signal simulating a pressure transducer signal readable by said any one of a plurality of vital signs monitors.

6. A system according to claim 5, wherein said pressure transducer emulation circuitry further includes a load adjustment circuitry for controlling current loads drawn by said pressure transducer emulation circuitry from said any one of a plurality of vital signs monitors.

7. A system according to claim 4, wherein said pressure transducer emulation circuitry includes a power harvesting circuitry for deriving power to drive said pressure transducer emulation circuitry from an excitation voltage signal from said any one of a plurality of vital signs monitors.

8. A medical system for transmitting pressure data to a monitor comprising:
a pressure transducer providing an analog transducer signal;
a portable unit connected to said pressure transducer, said portable unit comprising:
a power source supplying an excitation voltage to said pressure transducer;
an analog-to-digital module converting said analog transducer signal to a digital transducer signal; and
a portable wireless transceiver transmitting said digital transducer signal; and
a monitor interface unit connectable to a monitor, said monitor interface unit comprising:
a monitor wireless transceiver receiving said digital transducer signal from said portable wireless transceiver; and
a transducer emulation module converting said digital transducer signal to an emulated analog transducer signal;
wherein said emulated analog transducer signal communicates a pressure value measured by said pressure transducer to said monitor.

9. The medical system of claim 8, wherein said monitor is configured to recognize an input voltage in which about five microvolts of signal per volt of said input voltage is equal to about one millimeter of mercury applied pressure.

10. The medical system of claim 8, wherein said monitor interface unit includes a power supply harvester drawing power from a monitor excitation signal.

11. The medical system of claim 8, wherein said transducer emulation module includes a digital-to-analog converter circuit for generating an analog signal based on a said digital transducer signal and a reference voltage .

12. The medical system of claim 11, wherein said transducer emulation module includes an active bridge drive circuit for generating an bridge voltage based on said analog signal.

13. The medial system of claim 12, wherein said transducer emulation module includes a synthetic bridge circuit for generating a differential pressure voltage signal based on said bridge signal.

14. The medical system of claim 8, wherein said transducer emulation module includes a load adjustment circuit for adjusting a load placed on said monitor by said transducer emulation module.

15. The medical system of claim 8, wherein said portable unit further comprises a microcontroller adapted to encode said digital transducer signal into a transmission data packet.

16. A method of monitoring blood pressure of a patient comprising:
exciting a pressure transducer to generate an analog signal representative of a blood pressure of said patient;
converting said analog signal to a digital signal;
wirelessly broadcasting said digital signal from said patient;
receiving said digital signal at a monitor spaced from said patient;
processing said digital signal so as to generate an analog signal based on an excitation voltage format generated by said monitor and so as to generate a blood pressure signal compatible with said excitation voltage format;
communicating said blood pressure signal to said monitor.

17. A method according to claim 16, wherein said exciting of said pressure transducer includes exciting said pressure transducer with an energy source independent of said monitor.

18. A method according to claim 16, wherein the receiving, processing and communicating is performed with power provided by an excitation voltage signal from said monitor.

19. A method according to claim 16, wherein said blood pressure signal compatible with said excitation voltage format is a BP22 signal.

20. A method according to claim 16, wherein said processing of said digital signal includes:
generating a voltage reference signal representative of an excitation voltage of said monitor;
converting said digital signal into a first analog signal based on a value of said digital signal and said voltage reference signal;
generating a bridge signal from said first analog signal;
generating an analog differential voltage signal from said bridge signal;
providing said analog differential voltage signal to said monitor.

21. A method of wirelessly transmitting pressure data comprising:
providing a portable unit connected to a pressure transducer;
providing a first excitation signal to said pressure transducer;
receiving an output signal from said pressure transducer with said portable unit;
converting said output signal to a digital pressure value;
wirelessly transmitting said digital pressure value to. a monitor interface unit;
receiving a second excitation signal from a monitor;
creating an emulated pressure transducer signal based on said digital pressure value and said second excitation signal; and
supplying said emulated pressure transducer signal to said monitor.

22. The method of claim 21, wherein said providing a first excitation signal to said pressure transducer includes providing a battery connected to a power supply within said portable unit.

23. The method of claim 21, wherein said converting said output signal to a digital pressure value is followed by encoding said digital pressure value into a transmission data packet.

24. The method of claim 21, wherein said receiving a second excitation signal from a monitor includes powering said monitor interface unit with said excitation signal.

25. The method of claim 21, wherein said creating an emulated pressure transducer signal based on said digital pressure value includes modifying said second excitation signal based on said digital pressure value.

26. The method of claim 25, wherein said modifying said second excitation signal based on said digital pressure value includes providing said digital pressure value and a reference voltage to a digital to analog converter circuit.

27. The method of claim 25, wherein said receiving a second excitation signal from a monitor includes powering said monitor interface unit with said second excitation signal.

28. The method of claim 27, wherein said receiving a second excitation signal from a monitor interface unit includes monitoring a load by said monitor interface unit on said monitor and adjusting said load to maintain a predetermined load amount on said monitor.

29. The method of claim 21, wherein said creating an emulated pressure transducer signal based on said digital pressure value includes creating an emulated pressure transducer signal compliant with a BP22 standard.

30. The method of claim 21, further comprising:
repeating the wireless transmission of said digital pressure value so as to transmit a pressure wave form.

31. A pressure transducer telemetry system comprising:
a first unit sized and shaped to move with a patient, including a power source configured to provide a first excitation voltage to a pressure transducer, an analog-to-digital circuit coupled to receive a transducer signal and produce a digital pressure value based on said transducer signal, and a first wireless transceiver coupled to transmit said digital pressure value; and
a second unit connectable to multiple types of monitors, said second unit including a second wireless transceiver configured to wirelessly receive said digital pressure value, and a pressure transducer emulation circuit coupled to receive said digital pressure value and produce an emulated pressure transducer signal based on said digital pressure value, said emulated pressure transducer signal being readable by said multiple types of monitors;
wherein said second unit communicates said emulated pressure transducer signal to at lease one of said multiple types of monitors to display a pressure measurement.

32. The pressure transducer telemetry system of claim 31, wherein said first unit includes a battery.

33. The pressure transducer telemetry system of claim 31, wherein said pressure transducer emulation circuit includes a digital-to-analog converter circuit.

34. The pressure transducer telemetry system of claim 31, wherein said pressure transducer emulation circuit is further configured to maintain a predetermined load on said monitor.

35. The pressure transducer telemetry system of claim 31, wherein said digital-to-analog converter circuit modifies the voltage of a reference signal based on said digital pressure value.

36. The pressure transducer telemetry system of claim 31, wherein said at least one of said multiple types of monitor can determine a pressure reading according to a BP22 standard.

37. The pressure transducer telemetry system of claim 31, wherein said second unit further comprises a cable configured to connect to pressure transducer port on said at least one of said multiple types of monitor.

38. The pressure transducer telemetry system of claim 31, further comprising a second digital-to-analog converter connected to said power source so as to generate said first excitation voltage.

39. The pressure transducer telemetry system of claim 38, further comprising a second analog-to-digital converter connected to receive a second excitation voltage from said multiple types of monitors.

40. The pressure transducer telemetry system of claim 39, wherein said second digital-to-analog converter is configured to produce said first excitation voltage based on a digital value generated by said second analog-to-digital converter.

41. A pressure transducer telemetry system comprising:
a portable pressure transducer unit movable with a patient;
said portable pressure transducer unit generating a digital pressure value based on a pressure sensed by a pressure transducer;
a first wireless transceiver disposed on said portable pressure transducer unit for transmitting said digital pressure value;
a stationary unit connectable to an interface unit;
a second wireless transceiver disposed on said stationary unit for receiving said digital pressure value;
an interface unit disposed between said stationary unit and a vital signs monitor;
a conversion routine disposed in one of said stationary unit and said interface unit to convert said digital pressure value into a signal readable by said vital signs monitor.

42. A pressure transducer telemetry system according to claim 41, wherein said conversion routine is disposed in said stationary unit.

43. A pressure transducer telemetry system according to claim 41, wherein said conversion routine is disposed in said interface unit.

44. A pressure transducer telemetry system according to claim 41, said stationary unit includes multiple conversion routines for generating a signal readable by multiple types of vital signs monitors.

45. A pressure transducer telemetry system according to claim 44, wherein said stationary unit includes a selector for enabling a user to select which conversion routine to utilize.

46. A pressure transducer telemetry system comprising:
a portable pressure transducer unit movable with a patient;
said portable pressure transducer unit generating a digital pressure value based on a pressure sensed by a pressure transducer;
a first wireless transceiver disposed on said portable pressure transducer unit for transmitting said digital pressure value;
a stationary unit connectable to a vital signs monitor;
a second wireless transceiver disposed on said stationary unit for receiving said digital pressure value;
an interface circuit disposed within said stationary unit;
a communication protocol disposed in said interface circuit to communicate said digital pressure value to said vital signs monitor.

47. The pressure transducer telemetry system of claim 46, wherein said vital signs monitor includes a communication bus.

48. The pressure transducer telemetry system of claim 47, wherein said communication protocol communicates data over said communication bus.

49. The pressure transducer telemetry system of claim 46, wherein said vital signs monitor includes user inputs for controlling said stationary unit with said communication protocol.

50. The pressure transducer telemetry system of claim 46, wherein said stationary unit communicates a wireless signal strength of said first wireless transceiver to said vital signs monitor.

51. The pressure transducer telemetry system of claim 46, wherein said stationary unit communicates a battery level of said portable pressure transducer unit to said vital signs monitor.
